(19) United States
(12) Patent Application Publication
(10) Pub. No.: US 2013/0109997 A1
(43) Pub. Date: May 2, 2013

(54) SYSTEM FOR MONITORING BIOLOGICAL DATA

(71) Applicants: Peter Linke, Dallas, TX (US); Eva Zeisel, Lewisville, TX (US)

(72) Inventors: Peter Linke, Dallas, TX (US); Eva Zeisel, Lewisville, TX (US)

(21) Appl. No.: 13/287,851

(22) Filed: Nov. 2, 2011

(51) Int. Cl. A61B 5/01; A61B 5/00

(52) U.S. Cl. 600/549; 600/300

(57) ABSTRACT

A system and method configured to monitor biological data. The system includes a biosensor assembly for processing biological data of a living body. The biosensor assembly comprises a sensor configured to be coupled to a wearable item, for instance a cap worn on the head of a user. The sensor is positioned on the wearable item to gather raw biological data from the body of the user, when the wearable item is worn on the body of the user. The assembly further comprises a controlling unit configured to interpret raw biological data received from the sensor and interpret raw biological data by computing a value representative of a physiological condition of the body of the user. The controlling unit compares the representative value to a threshold value stored in memory to characterize the physiological condition of the user and alerts the user.

### BACKGROUND OF THE INVENTION

1. Field of the Invention

The present invention relates to the design of biological data monitors.

2. Description of the Related Art

According to Centers for Disease Control and Prevention (CDC) during 1999-2003, a total of 3,442 deaths resulting from exposure to extreme heat were reported (annual mean: 688). Many heat-related deaths are preventable with early detection. A need, therefore, exists for a system and method for monitoring the physiological condition of persons, particularly in monitoring body temperatures and other biological data.

### SUMMARY OF THE INVENTION

The present invention provides for a system and method configured to monitor biological data gathered by a sensor positioned on a living body. The system comprises a a controlling unit worn on the body and configured to interpret raw biological data received from the sensor and configured to alert the user of a physiological condition of the body.

### BRIEF DESCRIPTION OF THE DRAWINGS

For a more complete understanding of the present invention and the advantages thereof, reference is now made to the following Detailed Description taken in conjunction with the accompanying drawings, in which:

FIG. 1 is a schematic representation of an embodiment of a first system for monitoring biological data;

FIG. 2 is a schematic representation of an embodiment of a first system for monitoring biological data showing a biosensor assembly affixed to the inner portion of a cap;

FIG. 3 is a schematic representation of a first embodiment of a biosensor assembly;

FIG. 4 is a schematic representation of an embodiment of a second system for monitoring biological data;

FIG. 5 is a schematic representation of a second embodiment of a biosensor assembly;

FIG. 6 is an exploded view of a sensor pad assembly for use in a biosensor assembly;

FIG. 7 is a side view of a sensor pad assembly, showing layers of the sensor pad assembly;

FIG. 8 is a first perspective view of a wearable item, specifically a cap, showing a biosensor assembly coupled on inside portions of the cap;

FIG. 9 is a second perspective view of a wearable item, specifically a cap, showing a biosensor assembly coupled on inside portions of the cap;

FIG. 10 is a third perspective view of a wearable item, specifically a cap, showing a portion of a biosensor assembly being partially removed by a hand;

FIG. 11 is a block diagram of an embodiment of a first system for monitoring biological data showing signal flow between the components of the system;

FIG. 12 is a block diagram of an embodiment of a second system for monitoring biological data showing signal flow between the components of the system; and

FIGS. 13A, 13B, and 13C are flow charts showing operations in a process for monitoring biological data.

### DETAILED DESCRIPTION

In the following discussion, numerous specific details are set forth to provide a thorough explanation. However, such specific details are not essential. In other instances, well-known elements have been illustrated in schematic or block diagram form. Additionally, for the most part, specific details within the understanding of persons of ordinary skill in the relevant art have been omitted.

### I. The Biosensor Assembly 101 of the Systems 100, 100′

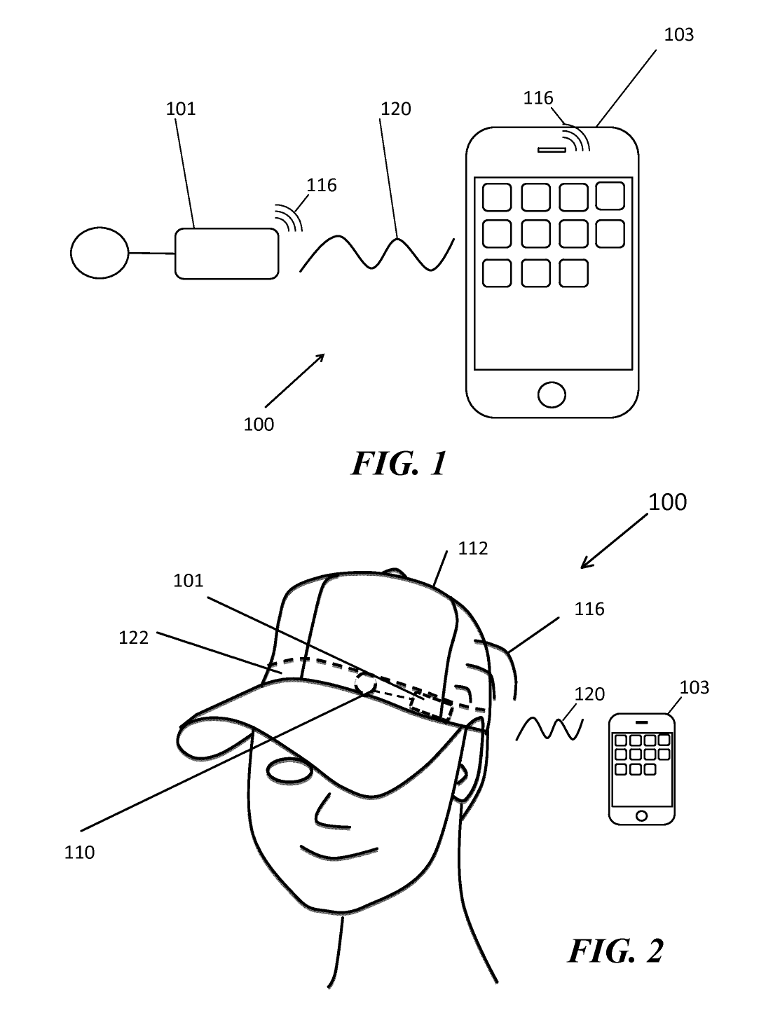

Referring now to FIG. 1, there is shown one embodiment of a system 100 configured to monitor biological data. The system 100 may comprise one or more biosensor assemblies 101. Each biosensor assembly 101 may be configured to capture, detect, and/or gather raw biological data characterizing physiological parameters of a living body (such as but not limited to shock, heart rate, temperature, dehydration or other raw biological data known to persons of ordinary skill in the art). The biosensor assembly 101 may be positioned proximal to the body to make operational contact with the body. It will be understood that operational contact may include direct contact with the skin of the body, or indirect contact (e.g. contact through protective or conductive layers, or contact through media such as air (e.g. detecting heat radiated from the body over a distance).

In some embodiments, the biosensor assembly 101 interprets raw biological data, for example, by making comparisons of the raw data or representative data based on raw data with threshold values stored in memory. The biosensor assembly 101 may convey to a user of the biosensor assembly 101 information relating to the physiological condition of the living body. In some embodiments information is communicated through an alert 116, which may communicate to the user or other authorized person information related to the interpretation of the biological data made by the biosensor assembly 101. For example, based on a comparison of the biological data, or data related to or derived from the biological data, with threshold values stored in memory, the biosensor assembly 101 may determine that a physiological parameter is abnormal, like a high body temperature or a fast heart rate.

Referring to FIG. 2, the system 100 may further comprise a wearable item, such as a cap 112 as shown, a chest strap (not shown) or other suitable garment, strap or fixing means for positioning the biosensor assembly 101 on the body of an intended wearer. The biosensor assembly 101 may be coupled to the cap 112 for positioning the biosensor assembly 101 in operational contact or proximal to the living body, when the cap 112 is worn, for capturing, detecting, and/or gathering raw biological data. The biosensor assembly 101 may be further configured to interpret the raw biological data and may transmit a response signal to alert the wearer or other authorized person of a physiological condition of the body of the wearer.

Referring again to FIG. 1, the system 100 may include a secondary device 103, operating in conjunction with the biosensor assembly 101. The secondary device 103 may generate the alert 116 based on the interpretation of the raw biological data. In some embodiments, the alert 116 may be a sound, like a beep, generated from a speaker indicating that the body has reached a threshold temperature.

The secondary device 103 may be any compatible device capable of communicating wirelessly along a path 120 with the biosensor assembly 101. The secondary device 103 may provide display capabilities and run a system software application for processing and output of raw biological data or data related to or derived from raw biological data. The secondary device 103 may comprise, for instance, a smart phone, tablet computer, laptop or desktop computer, PDA, an MP3 player (e.g. the Apple® Ipod® produced by Apple Inc.) or other computing device known by persons of ordinary skill in the art.

The alert 116 may be triggered by the response signal and communicated to the secondary device 103 wirelessly along the path 120. The alert 116 may comprise a sound, a display, a vibration, or other stimulus configured to communicate information to users of the secondary device 103.

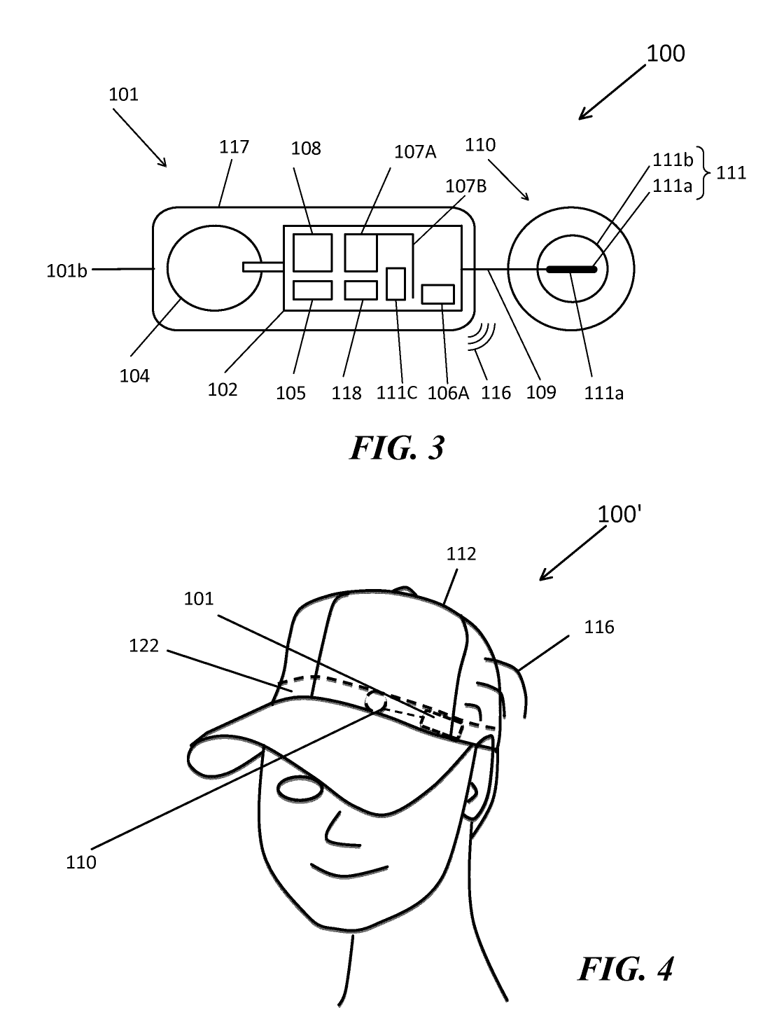

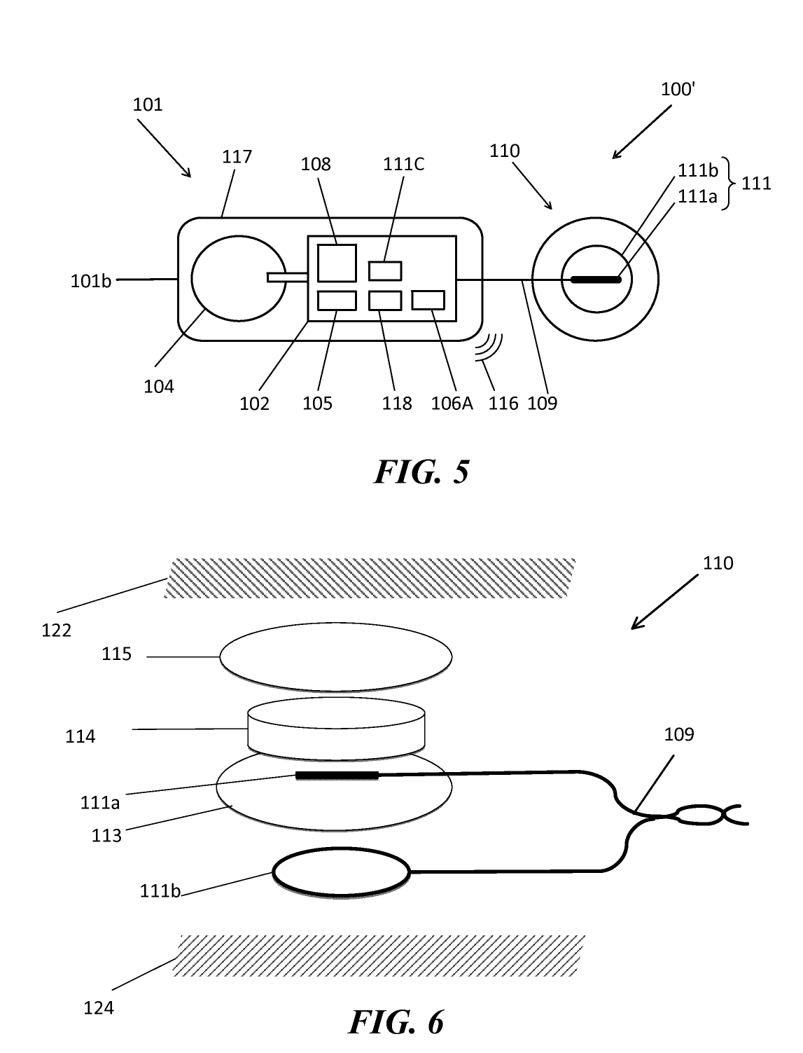

Referring to FIG. 4, there is shown a second system 100′ for monitoring biological data. The system 100′ may operate with or without incorporation of the secondary device 103. As shown in FIG. 3, the biosensor assembly 101 may generate the alert 116, without sending a signal to any other device located apart from the biosensor assembly 101, such as the secondary device 103 shown in FIGS. 1 and 2. The system 100′ may also operate with the embodiment of the biosensor assembly 101 shown in FIG. 5, which excludes certain hardware in biosensor assembly 101 shown FIG. 3. The system 100′ may be configured to stand-alone by interpreting biological data and informing the wearer, through alerts, about a physiological condition of the body of the wearer.

Referring to FIG. 4, the system 100′ may further comprise a wearable item, such as the cap 112 for attachment of the biosensor assembly 101. The wearable item (e.g. cap 112) may be substantially similar to that shown and described in FIG. 2.

### II. The Sensor Pad Assembly 110

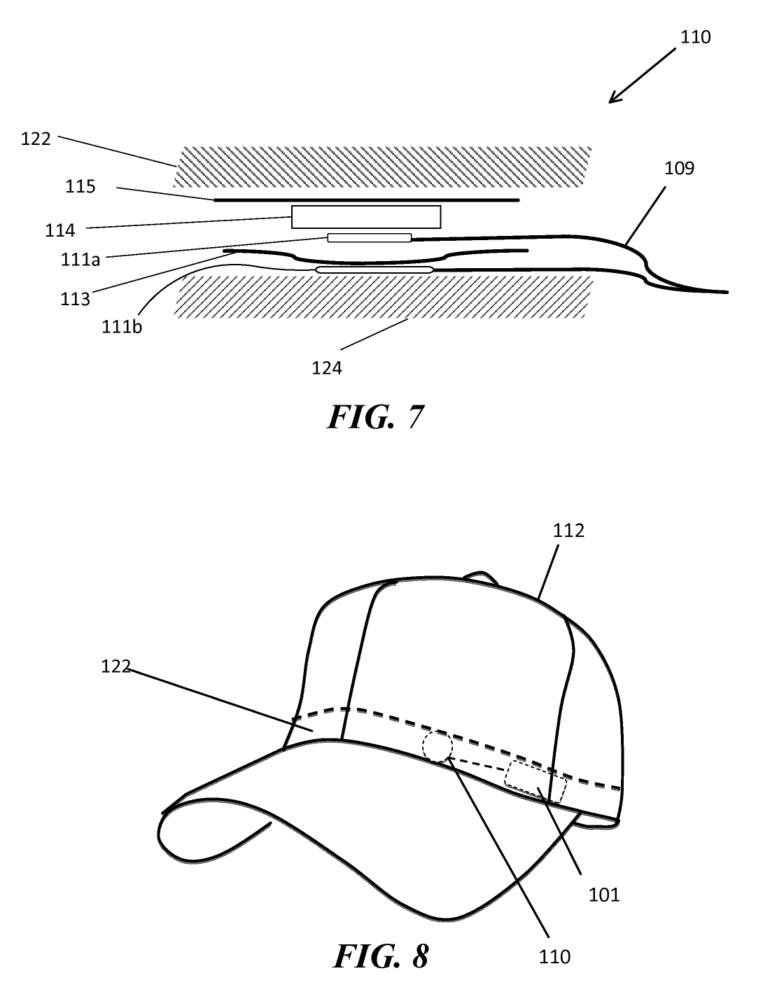

Referring now to FIGS. 6 and 7, there is shown two views of the structural layers of a sensor pad assembly 110 of the biosensor assembly 101. The sensor pad assembly 110 may comprise at least a part of a sensor unit 111. A portion of the sensor pad assembly 110 may be configured to directly make contact with a skin portion 124 of the body. In other embodiments, the sensor pad assembly 110 may not directly touch the skin 124, but may be positioned on the wearable item 112 to be proximal to the body for taking raw biological data, and maintain operational contact.

The sensor pad assembly 110 may include at least a portion of a sensor unit 111 comprising one or more biological data capturing devices, such as a temperature sensor 111a, a presence sensor 111b and, in some embodiments, other types of sensors. The temperature sensor 111a and the presence sensor 111b may be mounted or affixed to the sensor pad 110 so that portions of the sensor pad 110 support the temperature sensor 111a and the presence sensor 111b. The temperature sensor 111a may comprise a thermistor, a thermal ribbon type sensor or other type of temperature sensor.

The presence sensor 111b may be either a resistive or capacitive electrical sensor, proximity sensor or other suitable sensor capable of detecting that the wearable item is no longer being worn by the body. In other embodiments, the presence sensor 111b may be capable of detecting that the sensor unit is positioned proximal to the body to capture raw biological data.

Other types of sensors may be used in place of or in addition to either one or both of the temperature sensor 111a and the presence sensor 111b. Such other types of sensors may include a shock sensor, such as an accelerometer, and/or a heart rate sensor, such as electrical or optical sensors that measure blood flow pulses through blood vessels of the body. In some embodiments, these other types of sensors may be mounted in conjunction with the temperature sensor 111a and the presence sensor 111b. In some embodiments, these other sensors may be mounted on other portions of the biosensor assembly 101.

### III. Layers of the Sensor Pad Assembly 110

Referring again to FIGS. 6 and 7, the sensor pad assembly 110 may comprise one or more layers. A contact layer 113 may be configured to directly contact or remain proximal to the skin portion 124 of the body. The contact layer 113 may comprise a layer of material, which may be formed as a thin sheet and manufactured from a flexible plastic or metallic material, such as thermoplastic urethane (TPU), polyvinylchloride or other known suitable plastic, metallic or other material, which conducts heat and electricity.

On top of this layer or integrated inside the contact layer 113 is the presence sensor 111b. FIG. 7 shows the presence sensor 111b coupled on one side of the contact layer 113. The presence sensor 111b may be coupled on one side of the contact layer 113 by bonding the presence sensor 111b through heat, chemical, or adhesive methods. The presence sensor 111b may be also integrated below the contact layer 113 (not shown), depending on the type presence sensor 111b. Such integration may be accomplished by weaving the presence sensor 111b through a fabric portion of the contact layer 113, or embedding the presence sensor 111b beneath a cover portion of the contact layer 113.

A padding layer 114 may be coupled to the contact layer 113. The padding layer 114 may be manufactured from foam or other suitable material and positioned between the contact layer 113 and the temperature sensor 111a to protect the temperature sensor 111a from shock, moisture, and other environmental conditions that might disturb the temperature sensor 111a and/or compromise biological data gathering. The contact layer 113 may be glued (or otherwise bonded) to one side of the padding layer 114.

An adhesive layer 115 may hold the temperature sensor 111a to the padding layer 114 and also may be used for attachment to the wearable item 112. The adhesive layer 115 may comprise a sheet of adhesive paper or plastic having adhesive on one side, with the adhesive of the sheet holding the temperature sensor 111a to the padding layer 114.

### IV. Attachment of the Biosensor Assembly 101 to the Wearable Item (the Cap 112)

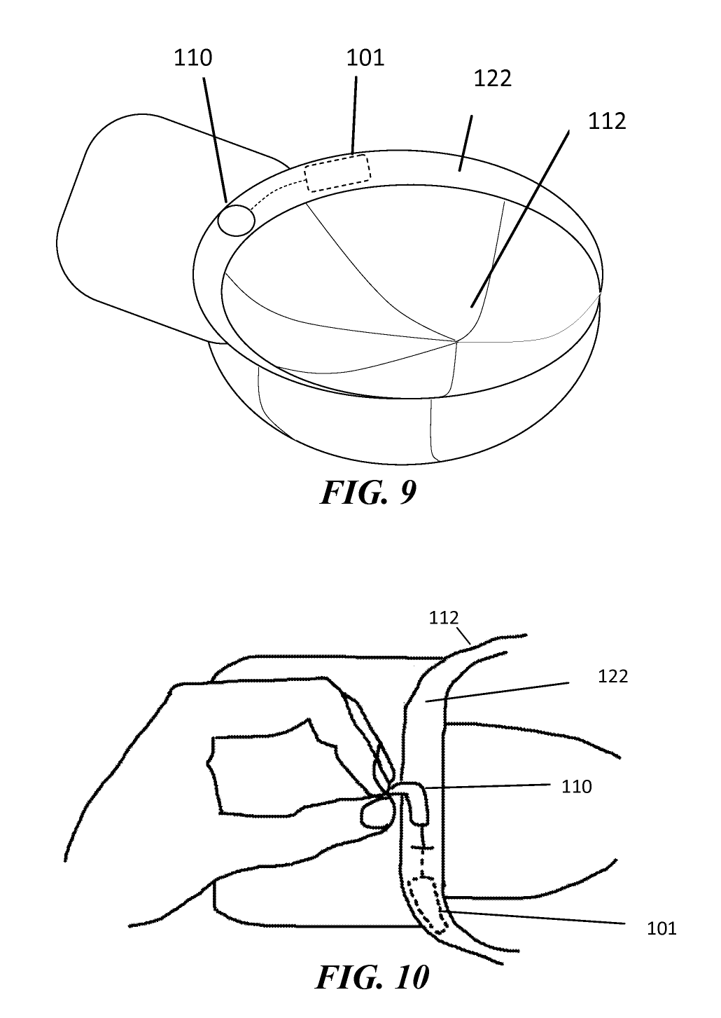

Referring to FIGS. 8 and 9, the sensor pad assembly 110 may be attached to the cap 112 using a fastener, such as adhesive or a fabric hook-and-loop fastener (e.g. the Velcro® brand) to allow removal or replacement of the sensor pad assembly 110. It will be understood that other fastening means known and apparent to persons of ordinary skill in the art may be used as fasteners, including clips, hooks, snap fits, buttons, and zippers, or other fasteners configure to provide attachment and removal of the biosensor assembly 101. The biosensor assembly 101 may be affixed to the cap 112 in a manner that positions the sensor pad assembly 110 relative to the living body for capturing raw biological data.

In embodiments using adhesive, the sensor pad assembly 110 may be coupled to the wearable item 112 by attaching the wearable item 112 to the opposite side of the adhesive layer 115 from the side glued to the padding layer 114.

In some embodiments, the opposite side of the adhesive layer 115 does not have adhesive, but is coupled to the wearable item through attachment means readily known in the art, such as use of Velcro, snap fits, buttons, and zippers.

In some embodiments, the sensor pad assembly may be attached to the cap 112 by a permanent fastener, such integrating the sensor pad assembly 110 directly into the fabric of the cap 112 or using permanent glue as an adhesive.

In some embodiments, the temperature sensor 111a and presence sensor 111b may be attached to the cap 112 by integrating the temperature sensor 111a and presence sensor 111b directly into the fabric of the cap 112, and without using one or both of the contact layer 113 and/or the padding layer 114. In these embodiments, the fabric of the cap 112 may function to protect the sensors 111a, 111b from environmental conditions. If no contact layer 113 and/or padding layer 114 are used, the temperature sensor 111a and presence sensor 111b may be directly integrated into the fabric.

The PCB 102 and the battery 104 may be attached on the inside of the cap 112 between the cloth comprising the sweatband 122 of the head covering and the crown of the head so that the cloth comprising a sweatband 122 comprises a barrier between the skin portion 124 of the wearer and the biosensor providing protection and padding for added comfort to the wearer.

The cable 109 may be threaded through or around the sweatband 122 to connect the sensor pad 110 to the biosensor assembly 101. The sensor pad assembly 110 is attached to the outside of the sweatband 122, directly touching the skin 124 of the wearer.

Referring to FIG. 10, the sensor pad assembly 110 in some embodiments can be removed by peeling off the sensor pad assembly 110 from the sweatband 122 or other surface of attachment. The sensor pad assembly 110 is then threaded through the opening in the sweatband 122 back towards the biosensor assembly 101 and the whole biosensor assembly 101 can be removed, as well.

### V. The Programmable Circuit Board 102 and Battery 104

Referring back to FIG. 3, there is shown one embodiment of the biosensor assembly 101 operable in systems 100 and 100′. The temperature sensor 111a and the presence sensor 111b attached may be operationally connected via an electrical path, such as a cable 109, to a programmable circuit board (“PCB”) 102 of the biosensor assembly 101. The sensors 111a and 111b may send biological data to a controlling unit 108, where it is received and interpreted. It will be understood by persons of ordinary skill in the art that the cable 109, shown in FIGS. 5 and 6 may provide an electrical path for transfer of data signals between sensors 111a, 111b in the sensor unit and the PCB 102.

The PCB 102 may also include other sensors 111c (such as a shock sensor, or other sensor configured to capture biological data) that are located on-board the PCB 102 (see also FIG. 5 showing the sensors 111c in the PCB 102 of the alternative embodiment of biosensor assembly 101). These other sensors 111c may form a part of the sensor unit 111 and be configured to provide biological data to the controlling unit 108 for monitoring biological data of the living body.

It should be understood by persons of ordinary skill in the art that the sensors of the sensor unit 111, for example the temperature sensor 111a and the presence sensor 111b, may be integrated onto the PCB 102, instead of being distanced by the length of the cable 109. The length of the cable 109 may be shortened until effectively the sensor unit 111, comprised of sensors 111a, 111b, and 111c are entirely integrated onto the PCB 102.

The PCB 102 may comprise the controlling unit 108, a wireless communication transceiver 107a with an antenna 107b, which may also be a separate transmitter and receiver, a memory device 105 and an on-board signaling device 106a, such as a beeper or other types of human interface devices, including visual lights, visual display, and/or vibration devices.

The transceiver 107a and the antenna 107b may comprise a communication device configured to communicate with the secondary device 103 via a wireless network. The wireless protocol used by the biosensor assembly 101 (of either FIG. 3 or 5) for communication with external devices may comprise one or more of Wi-Fi® (a trademark of Wi-Fi Alliance, Austin, Tex., USA), Bluetooth® (a trademark of Bluetooth SIG, Kirkland Wash., USA), Radio Frequency Identification (RFID), cellular (for example third generation mobile technology (3G), fourth generation mobile technology (4G), and 3GPP Long Term Evolution (LTE)) or other wireless communication protocols or wireless technology standards suitable and known to persons of ordinary skill in the art.

As shown in FIGS. 1 and 2, the biosensor assembly 101 may communicate wirelessly over the signal path 120 with the secondary device 103. The secondary device 103 may comprise a smart phone, PDA, tablet, or other computing device capable of receiving and interpreting the wireless signals of the biosensor assembly 101. The wireless communication between the biosensor assembly 101 and the secondary device 103 may be performed using a wireless protocol, as described above.

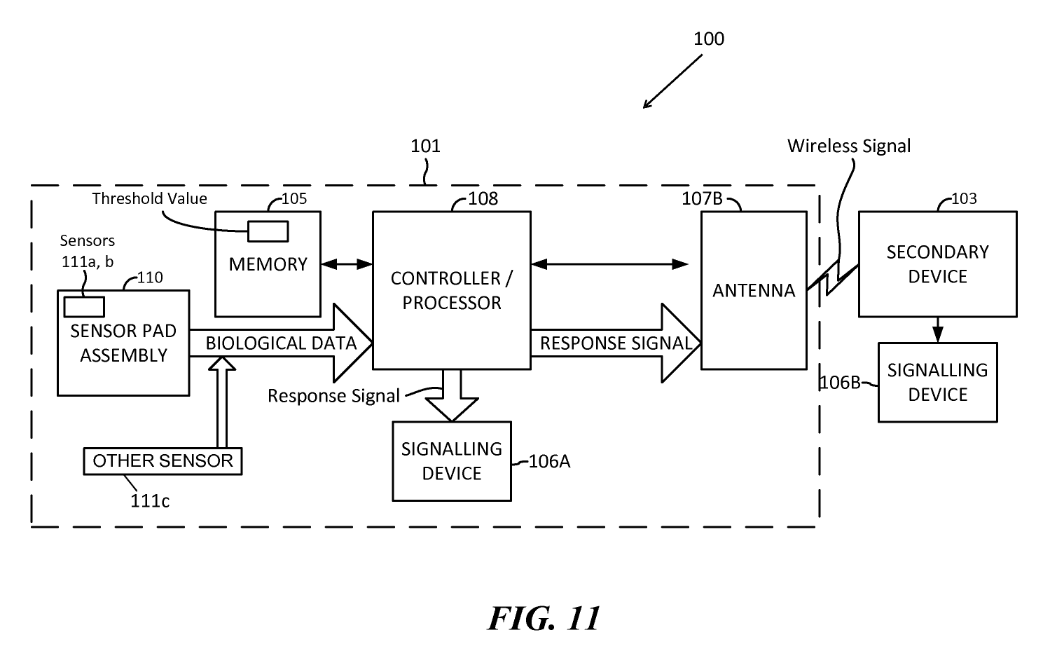

Turning now to FIG. 11, there is shown a schematic representation of hardware of the system 100, indicating the communication of signals between the different components. The controlling unit 108 may comprise a controller or a processor configured to interpret raw biological data received from the sensors 111a, 111b. Interpretation of raw biological data may include data validation (i.e. rejection of erroneous or corrupted data), data filtering based on settings (i.e. stripping data with values lower than certain minimum threshold and higher than certain maximum threshold), comparison with various thresholds for alerts, confirming that the biosensor assembly 101, for example analyzing data from the presence sensor 111b, is positioned to gather raw biological data, and other types of data analysis known by or apparent to persons of ordinary skill in the art. The controlling unit 108 may further communicate with the secondary device 103 by transmitting to, receiving from, and interpreting signals related to operation of the biosensor assembly 101 (i.e. battery status, time stamping, etc.).

The controlling unit 108 of the biosensor assembly 101 may compute a value representative of the raw biological data from the sensors 111a, 111b, and in some embodiments other sensor 111c. The representative value may correlate to a physiological condition of the living body; for example the biosensor assembly 101 may compute a body temperature. It will be understood by persons of ordinary skill in the art that the representative value computed by the controlling unit 108 may correlate to a variety of physiological conditions and may depend on the type of sensor capturing the biological data. For example, the representative value may comprise a rate of change of a physiological parameter with respect to time or other parameter, such as measuring the rate of change of temperature, pulse, or blood pressure over time.

The biosensor assembly 101 may compare the representative value with a preset or adjustable value stored in the memory 105 to further characterize a physiological condition of the body. For example, the controlling unit 108 may compare the body temperature of the living body with a preset or adjustable threshold value stored in the memory 105 to determine that the temperature of the body is relatively too high. Or the controlling unit 108 may detect a rapid rise in pulse, when it compares the change in pulse rate versus a threshold value stored in the memory 105.

The controlling unit 108 may detect whether the cap 112 has been put on the living body. The controlling unit 108 may receive data from the presence sensor 111b indicative of the presence of a living body, for example a signal representative of capacitance or resistance generated by the presence sensor 111b. The controlling unit 108 may interpret this data as indicating that a living body is proximal to the sensor pad assembly 110, and may infer that the sensor unit 111 is in a position to receive reliable raw biological data, for example that it is in operational contact.

As shown in FIG. 11, based on the interpretation of data received by the controlling unit 108 from the sensor unit 111 (sensors 111a, b, and c), the controlling unit 108 may generate a response signal. The response signal may include a command to the on-board signaling device 106a to issue an alert, for example an audible beep, to inform or warn the wearer that a threshold value has been exceeded. In embodiments where the signaling device 106b is located on the secondary device 103, the response signal may include a command to the transceiver 107a/antenna 107b to communicate with the secondary device 103 and deliver a wireless command to an external signaling device 106b to issue the alert 116.

It will be understood by persons of ordinary skill in the art that the PCB 102 will include other commonly known and used parts such as data input and output ports to support operation of the PCB 102 within the system 100 or the second system 101′. The data input and output ports 118 may be provided for downloading and uploading data, programming, firmware updates, and other information apparent for operation of the biosensor assembly 101. It will be understood by persons of ordinary skill in the art that such input and output ports 118 may comprise either wired or wireless ports for the exchange or transfer of data.

Referring back to FIG. 3 (and FIG. 5), the biosensor assembly 101 may include a power source, such as an internal battery 104 or the biosensor assembly 101 may utilize multiple batteries, solar cells, or other suitable power sources. The internal battery 104 may be operationally connected to the PCB 102 through an electrical path to the PCB 102. The biosensor assembly 101 may communicate the battery status to the user using audible or mechanical (vibration) alerts, such as the alert 116. In some embodiments, the battery status may be also communicated to the secondary device 103 in real-time, if the secondary device 103 is used during the period when biological data is captured, or after the data capture period if the data is uploaded to the secondary device 103 from the memory 105.

In some embodiments of system 100′, the biosensor assembly 101 may not include hardware for wireless communication, as shown in FIG. 5. The exclusion of such hardware, such as the antenna 107a and transceiver 107b, that is shown in FIG. 3, may simplify the on-board processing done within the controlling unit 108 shown in FIG. 5. In such embodiments, the secondary device 103 may not be used to communicate wirelessly with the biosensor assembly 101. The biosensor assembly 101 may comprise a stand-alone capability to interpret raw biological data, and convey information to the user of the biosensor assembly 101, through, for example, the alert 116 (shown in FIG. 4).

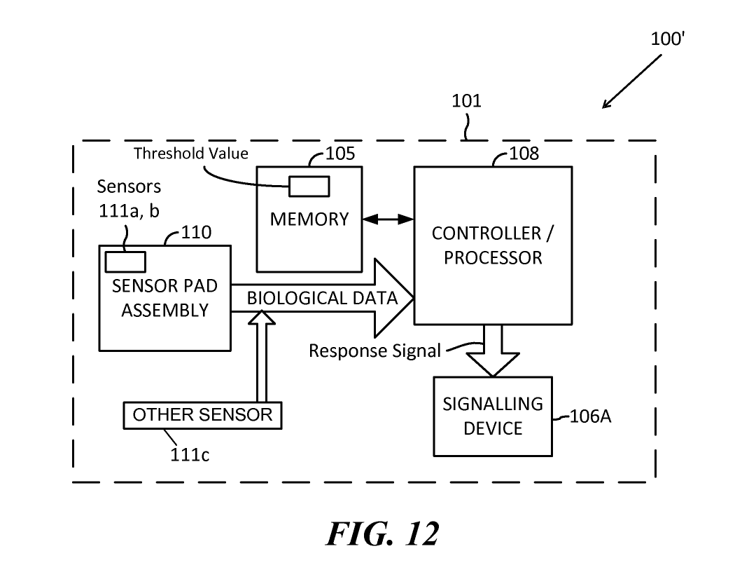

Referring to FIG. 12, there is shown a schematic of an embodiment of the biosensor assembly 101 of system 101′. The biosensor assembly 101 may exclude hardware for wireless communication, such as the antenna 107a and the transceiver 107b. The biosensor assembly 101 may operate in a manner similar to that described for the biosensor assembly 101 of system 100, except that system 100′ does not include a secondary device 103.

The on-board signaling device 106a may be affixed to the cap 112 and form a part of the biosensor assembly 101, as shown in FIGS. 3 and 5. The wearer of the cap 112 may transport the on-board signaling device 106a with the wearer so that the system 100′, shown in FIG. 4, may operate autonomously in interpreting data and conveying information relating to the biological data to the wearer, or in some cases, to authorized persons near the wearer.

In the other embodiments of the system 100, the transceiver 107a, the antenna 107b of the biosensor assembly 101 shown in FIG. 3, and the secondary device 103, shown in FIG. 2, may be disabled, as an option, by the wearer of the cap 112 or by the user of the secondary device 103. In these embodiments, the system 100 will operate in a manner similar to system 100′ for the period when the wireless communication hardware is disabled.

In some embodiments, the systems 100 or 100′ may not include the presence sensor 111b. It will be understood by persons of ordinary skill that the systems 100 or 100′ may incorporate other techniques, besides use of the presence sensor 111b designed to infer whether the cap 112 is being worn properly or whether the raw biological data gathered by the temperature sensor 111a, or other sensor, is reliable for characterizing the physiological condition of the body. For example, the controlling unit 108 may interpret an abnormally high or low temperature reading or pattern of readings as indicating by inference that the cap 112 is not on the wearer's body.

### VI. The Housing 117 of the Programmable Circuit Board 102 and Battery 114

Referring again to FIG. 3 (and similarly numbered components of FIG. 5), a portion of the biosensor assembly 101 may be configured with a housing 117 (also shown in FIG. 5 for system 101′) to resist water and other adverse environmental contaminants from entering the housing 117 or adversely affecting the PCB 102 and its components. In some embodiments the housing 117 of the biosensor assembly 101 may be waterproof having Ingress Protection (“IP”) of 67, referred to as IP 67. The housing 117 of the biosensor assembly 101 may comprise completely watertight enclosure that encapsulates and/or seals the PCB 102 and the internal battery 104.

The housing 117 may allow a user of biosensor assembly 101 of the system 100 (shown in FIG. 2) or the system 100′ (shown in FIG. 4) to wash the wearable item 112 without detaching the biosensor assembly 101 from the wearable item 112. The housing 117 may also allow use and wear of the item 112 in inclement weather (rain, snow, high humidity, etc.). It should be understood by persons of ordinary skill that the level of protection against environmental conditions may be varied according to the intended use of the system, and according to varied configurations, such as whether the housing 117 or sensor pad assembly 110 are removable from the wearable item (such as the cap 112, shown in FIGS. 2 and 4). The housing 117 may be manufactured from thermoplastic elastomer (TPE), Polycarbonate/Acrylonitrile Butadiene Styrene blend (PC/ABS) or other suitable plastic known and apparent to persons of ordinary skill in the art.

### VII. The Method 200 for Monitoring Biological Data

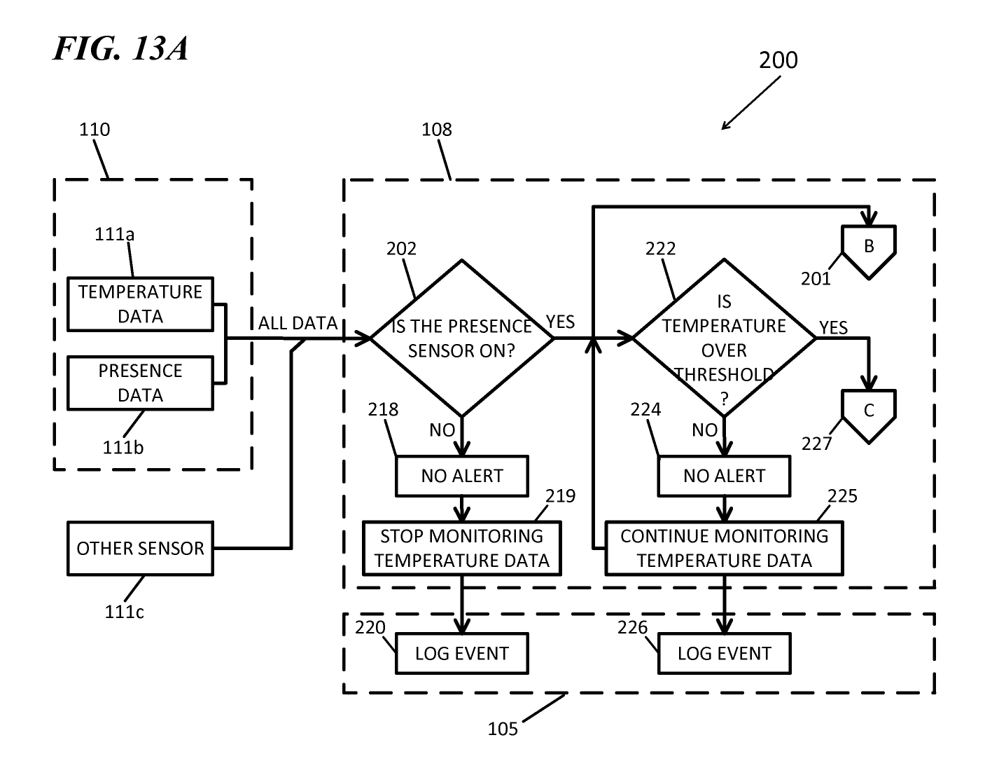

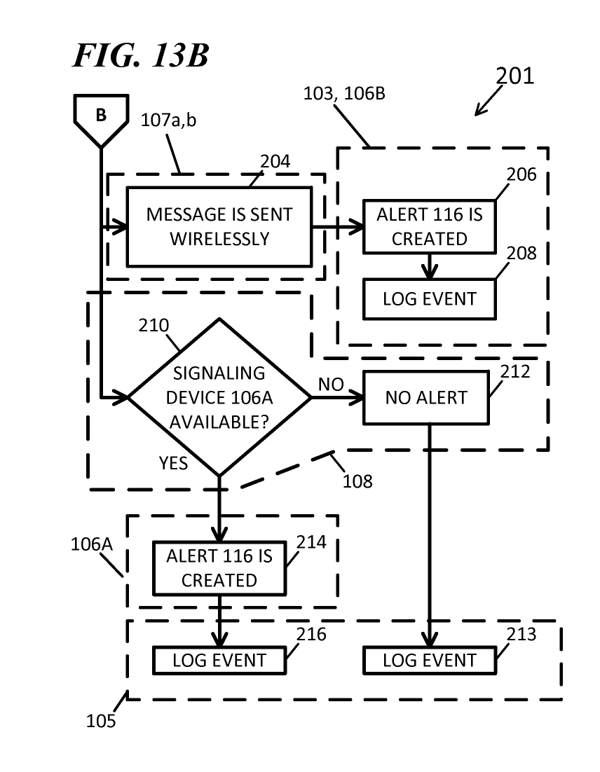

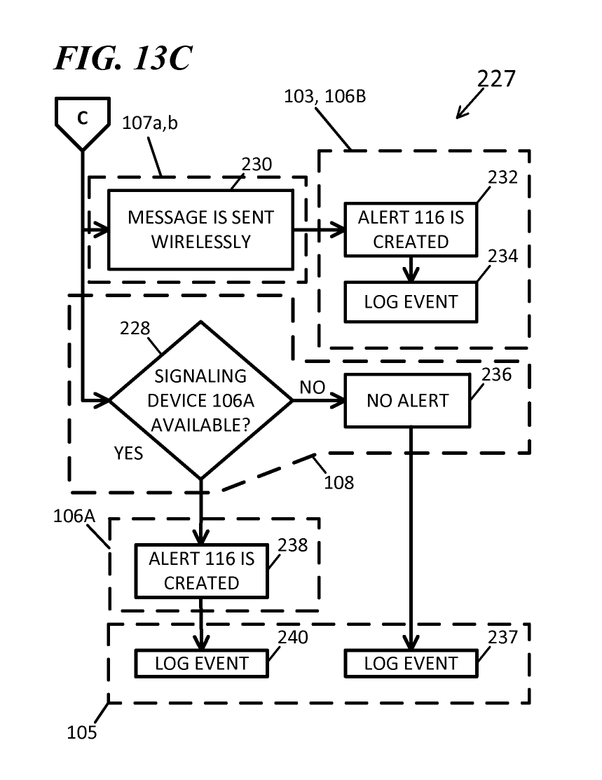

The systems 100 and 100′ shown and described above may be utilized in a method 200 configured to monitor biological data. The method 200 may comprise one or more operations shown in FIGS. 13A, 13B, and 13C.

An operational state of the biosensor assembly 101 may be controlled by switching the biosensor assembly between one or more modes. For example, the biosensor assembly 101 (shown in FIGS. 3 and 5) may comprise an “off” mode where power to the PCB 102 is cut off, and the PCB 102 cannot gather or interpret biological data. The off mode may be useful in conserving battery power.

The biosensor assembly 101 may comprise an “idle” mode where the biosensor assembly 101 is active and can send raw biological data to the PCB 102 for interpretation. In some embodiments, the biosensor assembly 101 may be activated by a wireless (or wired) signal sent to the PCB 102 at the point of manufacturing, or the PCB 102 may be pre-configured to be in the idle mode without requiring a wireless signal to activate the biosensor assembly 101. In other embodiments, the user may toggle the biosensor assembly 101 between the “off” mode and the “idle” mode through mechanical switches, wireless signals transmitted to the PCB 102, or other methods commonly known or apparent to persons of ordinary skill in the art.

The biosensor assembly 101 may comprise an “active” mode, where the biosensor assembly 101 is gathering raw biological data, and the PCB 102 is receiving such data for interpretation. The active mode may be triggered by a positive signal from the presence sensor 111b that the sensors 111a and/or other sensor 111c are in position to take reliable raw biological data.

Referring now to FIGS. 2 and 4, in order to start monitoring biological data, the user may put on the wearable item, such as the cap 112, on the head of the body, which may position the sensors 111a and/or other sensor 111c in position to take biological data, for example in operational contact with the body. The biosensor assembly 101 may be pre-set in the idle mode, allowing for detection of the presence of a wearer of the cap 112 and transition to the active mode, where biological data is monitored.

The cap 112 may be worn on the head so that presence sensor 111b detects that sensor unit 111 is positioned relative the body to take reliable raw biological data, for example that the sensor 111a is in operational contact with the body. In operation 202, shown in FIG. 13A, the controlling unit 108 may receive a signal from the presence sensor 111b indicating that the cap 112 is being properly worn by a living body.

As shown in the subprocess 201 shown in FIG. 13B, the controlling unit 108 may send a signal to either or both the on-board signaling device 106a or the external signaling device 106b located on the secondary device 103 that generates conveys information to the wearer or other authorized person near the wearer. In operation 204, the controlling unit 108, via the transceiver 107a and antenna 107b, sends a wireless signal to the secondary device 103 to generate the alert 116. In operation 208, the secondary device 103 may log the event in its internal memory for recovery at a later time. The information conveyed may be the alert 116, such as an audible beep or other type of signaling event (e.g. a message on a display of the secondary device 103) to the user of the secondary device 103.

In operation 210, the controlling unit 108 may determine whether the on-board signaling device 106a is available to generate the alert 116. The determination in operation 210 may depend on whether the biosensor assembly 101 includes an on-board signaling device 106a, or whether the user has disabled the on-board signaling device 106a, or whether the user has configured the controlling unit 108 to send a signal to both the secondary device 103 and the on-board signaling device 106a. The controlling unit 108 may determine that the on-board signaling device 106a is available, when the signaling device 106a is in an “on” state and is selected for generating alerts.

In some embodiments, the controlling unit 108 generates a signal to both the secondary device 103 and the on-board signaling device 106a, based on determinations made in operations 202 and 210. In operation 206, the secondary device 103 receives the command to generate the alert 116 at the external signaling device 106b. In operation 212, the controlling unit 108 may not send a signal to the on-board signaling device 106a based on the determination made in operation 210. For example, the controlling unit 108 may be configured to command only the external signaling device 106b to generate the alert 116.

In operation 214, the controlling unit 108 may send a signal to the on-board signaling device 106a to generate the alert 116. The creation of the alert 116 in operation 214 will depend on the determination in operation 210 that the signaling device 106a is available to generate alerts. For example, the controlling unit 108 may be configured to command only the on-board signaling device 106a to generate the alert 116, or the secondary device 103 may not be available for communication with the biosensor assembly 101.

In operation 216, the data relating to the event of operation 214 may be logged in the memory 105. Such data may include, but is not limited to, time stamps for alerts, sensor data logs, and communications logs between the biosensor assembly 101 and the secondary device 103.

Referring again to operation 202 in FIG. 13A, the controlling unit 108 may determine that the presence sensor 111b has not detected that a living body is wearing the cap 112 properly for gathering raw biological data. Or in some cases that the sensor unit 111 is not positioned on the wearer correctly to capture raw biological data. For example, the cap 112 may be misaligned on the head of the body so that the presence sensor 111b sends data to the controlling unit 108 which indicates that the raw biological data would not be reliable for interpreting a physiological condition of the body.

In operation 218, the controlling unit 108 may generate a signal to indicate that the presence sensor 111b has not detected that the cap 112 is being worn based on the determination in operation 202. In some embodiments, no alert may be generated by the signaling device 106a or external signaling device 106b as a result of the indication in operation 218. In other embodiments, the controlling unit 108 may issue a command to the signaling devices 106a, 106b to generate an alert to inform the potential wearer or a user of the secondary device 103 that the cap 112 is not being properly worn to take raw biological data. If no presence is detected, the biosensor assembly 101 may continue in the idle mode. In operation 220, the data relating to the event of operations 202, 210, and 218 may be logged in the memory 105. Such data may include, but is not limited to, time stamps for alerts, sensor data logs, and communications logs between the biosensor assembly 101 and the secondary device 103.

In operation 222, the biosensor assembly 101 may transition between the idle mode to the active mode, based on the indication in operation 202 that the presence sensor 111b has detected that the sensors 111a and/or 111c are in proper position to take reliable biological data, for example that the sensors 111a and/or 111c are in operational contact with the body. In the active mode, the controlling unit 108 may receive temperature data gathered from the living body by the temperature sensor 111a of the sensor unit 111 for interpreting the data. In some embodiments, the controlling unit 108 may compute a representative biological data value based on the temperature data. This value may represent a physiological condition of the body; for example, the controlling unit 108 may compute the body temperature of the wearer. If the wearer is exposed to hot conditions and/or performs physical activity, his/her temperature will likely rise.

As shown in FIG. 11, one or more preset or adjustable threshold values may be stored in the memory 105. In some embodiments, the threshold value may be adjusted via data ports 118 on the PCB 102. In other embodiments, the secondary device 103 may be used to communication with controlling unit 108 and adjust the threshold values stored in memory 105.

In operation 222, the controlling unit 108 may interpret the biological data received from the sensors 111a and/or 111c. In some embodiments, the controlling unit 108 may make one or more comparisons of the body temperature computed by the controlling unit 108 with the threshold value stored in member 105. It will be apparent to persons of ordinary skill in the art that the interpretation of raw biological data in operation 222 may comprise other analysis of the biological data that will characterize a physiological condition of the body.

In operation 224, the controlling unit 108 may generate a signal to indicate that the body temperature has not reached a threshold value. In some embodiments, no alert may be generated by the signaling device 106a or external signaling device 106b as a result of the indication in operation 224. In other embodiments, the controlling unit 108 may issue a command to the signaling devices 106a, 106b to generate an alert to inform the potential wearer or a user of the secondary device 103 that the body temperature has not reached a threshold value within a certain time interval.

In operation 225, the controlling unit 108 may continue to repeat operation 222 for further monitoring and interpretation of the body temperature of the wearer. The operation of continuing to monitor and interpret body temperature may be based on a further indication that the presence sensor 111b has detected that the sensors 111a and/or 111c are in proper position to take reliable biological data. Execution of successive instances of operation 222 may be separated by a preset or adjustable time interval.

Once the body temperature of the wearer, as determined in operation 222, reaches a threshold value established within the logic of the controlling unit 108, the controlling unit 108 may activate the signaling device 106a or 106b with a response signal to generate the alert 116.

As shown in the subprocess 227 shown in FIG. 13C, the controlling unit 108 may send a response signal to either or both the on-board signaling device 106a or the external signaling device 106b located on the secondary device 103 that generates the alert 116 to convey information to the wearer, other bystanders near the wearer, or the user of the secondary device 103. In operation 230, the controlling unit 108, via the transceiver 107a and antenna 107b, may send a wireless signal to the secondary device 103 to generate the alert 116 (operation 232) to inform the user of the secondary device that the body temperature of the wearer of the cap 112 has reached a threshold value. In operations 232 and 234, the secondary device 103 may generate an alert and log data related to the event in its internal memory for recovery at a later time. The information conveyed may be the alert 116, such as an audible beep or other type of signaling event (e.g. a message on a display of the secondary device) to the user of the secondary device 103.

Referring to operation 228 in FIG. 13C, the controlling unit 108 may determine whether the on-board signaling device 106a is available to generate the alert 116. The determination in operation 228 may depend on whether the biosensor assembly 101 includes an on-board signaling device 106a, or whether the user has disabled the on-board signaling device 106a, or whether the user has configured the controlling unit 108 to send a signal to both the secondary device 103 and the on-board signaling device 106a. The controlling unit 108 may determine that the on-board signaling device 106a is available, when it is in an “on” state and is selected, either by default programming or optionally by the user of the biosensor assembly 101, for generating alerts.

In some embodiments, the controlling unit 108 generates a signal to both the secondary device 103 (indicated in operation 230) and the on-board signaling device 106a, based on determinations made in operations 222 and 228. In operation 238, the controlling unit 108 commands an available on-board signaling device 106a to generate the alert 116 to inform the user or a nearby bystander of the determinations made in operations 222 and 228. In operation 240, the information related to the events of operation 238 may be logged in memory 105.

In operation 236, the controlling unit 108 may generate a signal to indicate that on-board signaling device 106a is not available to generate alerts. For example, the controlling unit 108 may be configured to command only the external signaling device 106b, and not the on-board signaling device 106a, to generate the alert 116. In some embodiments, no alert may be generated by the signaling device 106a. In operation 237, the controlling unit 108 may log into memory 105 information related to the events of operation 236.

Referring again to operation 222 in FIG. 13A, the controlling unit 108 may determine by comparison that the temperature of the body has not reached a threshold value stored in the memory 105. In operation 224, the controlling unit 108 may generate a signal to indicate that the temperature of the body has not reached a threshold value stored in the memory 105, based on the determination in operation 222. In some embodiments, no alert may be generated by the signaling device 106a or external signaling device 106b as a result of the indication in operation 224. In other embodiments, the controlling unit 108 may issue a command to the signaling devices 106a, 106b to generate an alert to inform the potential wearer or a user of the secondary device 103 that the temperature of the body has not reached a threshold value stored in the memory 105.

Following a first determination that the temperature of the body has not reached a threshold value stored in the memory 105 in operation 222, the controlling unit 108 may continue to monitor and re-execute operation 222, depending on the logic of the controlling unit 108. In operation 225 in FIG. 13A, the determination that the temperature of the body has not reached a threshold value may prompt the controlling unit 108 to continue monitoring.

In some embodiments, the monitoring function of the controlling unit 108 in operation 222 may be delayed by a preset or adjustable interval of time. The monitoring function in operation 222 may be interrupted by a determination by the controlling unit 108 based on data from the presence sensor 111b that the cap 112 is no longer being worn or that the sensor unit 111 is no longer positioned for receiving raw biological data, for example the cap 112 is only partially worn due to movement of the wearer. In operation 219 in FIG. 13A, the controlling unit 108 may issue a command to stop monitoring temperature data due to a determination in operation 202 that the presence sensor 111b has detected that the sensors 111a, 111c of the sensor unit 111 is not positioned to take reliable raw biological data, for example that the sensors 111a, 111c are no longer in operational contact with the body.

In some embodiments, there may be multiple threshold values stored in the memory 105 of the controlling unit 108. These threshold values may trigger different alerts 116 based on the information that the user is intended to receive from the alert 116 and based on a determination of how the raw biological data compares to the threshold value. For example, the alert 116 generated by the on-board signaling device 106a may become increasingly alarming based on a potentially harmful rise in body temperature.

In some embodiments, the biosensor assembly 101 may send a first audible signal as the alert 116 associated with a first threshold value (i.e. the cap is being worn properly and has detected a “normal” body temperature), and the biosensor assembly 101 may send a second audible signal as the alert 116 associated with a second threshold value (i.e. the temperature has reached “elevated” but still safe levels). The controlling unit 108 of the biosensor assembly 101 may send different sequences of audible signals based on the associated threshold value. For instance, if the biosensor assembly 101 detects a body temperature of over one hundred (100) degrees Fahrenheit, the controlling unit 108 of the biosensor assembly 101 may send a signal to the signaling device to execute one beep; if the biosensor assembly 101 detects a body temperature of over one hundred and one (101) degrees Fahrenheit, the controlling unit 108 of the biosensor assembly 101 may send a signal to the signaling device to generate the alert 116; if the biosensor assembly 101 detects a body temperature of over one hundred and two 102 degrees Fahrenheit, the controlling unit 108 of the biosensor assembly 101 may send a signal to the signaling device to generate further beeps.

Such elevation in the number of beeps as the detected body temperature rises may continue in a similar pattern, or in an increasingly noticeable or alerting pattern, until action is taken to address the rise in temperature. The alerts generated may be configured to communicate to the user or other authorized person that the health of the body of the user is at greater risk compared to a normal condition or compared to a prior alert. This example is presented for illustrative purposes only and is not intended to limit the usefulness of the biosensor assembly 101 in detecting other changes in biological parameters. It would be understood by persons of ordinary skill in the art that such thresholds may be set for other levels and different increments of increase or decrease (e.g. every 2 degrees decrease). It would be understood by and apparent to persons of ordinary skill in the art that thresholds for signaling the wearer or other persons may be implemented in the detection of other biological parameters, such as shock, heart rate, and blood pressure.

The alert 116 provided by the signaling device 106a or 106b of the biosensor assembly 101 to the user may not be only audible alerts; the alerts may also be vibrations (for example for the hearing impaired) generated by vibration device. Once the user receives the alert 116, he/she will know that his/her body temperature is rising. Such a rise in temperature may be an indication of that the body is overheating or that the body needs fluids and/or a cool down period in the shade, air-conditioned building, etc.

The controlling unit 108 may continuously receive data from the presence sensor 111b. The controlling unit 108 may repeat operation 202 continuously, or at preset or adjustable intervals, to monitor whether the cap 112 is being worn or whether the sensor unit 111 is in position to capture raw biological data, even when the biosensor assembly 101 is in the active mode of monitoring. When the cap 112 with the biosensor assembly 101 is taken off by the wearer and is no longer in operational contact with the body, the controlling unit 108 may receive data from the presence sensor 111b indicating that the controlling unit 108 may not be receiving reliable data from the temperature sensor 111a. In operation 219 in FIG. 13A, the controlling unit 108 may cease monitoring raw biological data and may no longer send alerts regarding the temperature. This avoids false or misleading alerts from the biosensor assembly due to high ambient temperature, when stored in hot environments (such as inside a car on a hot day), during washing, etc. and also extends the battery life of the biosensor assembly.

An RFID connection may be established between the biosensor assembly 101 and the secondary device 103. The secondary device 103 may include an RFID reader to receive and interpret the RFID signal from the biosensor assembly 101, in system 100 shown in FIGS. 1 and 2 for example.

### VIII. Use of the Systems 100 and 100′ and the Process 200

In embodiments where the biosensor assembly 101 is used together with the secondary device 103, such as the system 100 shown in FIGS. 1 and 2, the biosensor assembly 101 may send the temperature and other sensor data and battery status to the secondary device 103, which may run a software application capturing this data and providing the alert 116 based on preset or adjustable thresholds. The threshold values may be stored in a memory on the secondary device 103. The alert 116 generated by the external signaling device 106b (shown in FIG. 11) can be audible, visual or mechanical (vibration) and may be provided in real time. The software on the secondary device 103 may also provide historical reports of temperature and other measured biometrics and alert history.

In other embodiments, such as those shown for system 100′ in FIGS. 3 and 4, the biosensor assembly 101 may be used as a standalone device that stores captured raw biological data and stores it in memory, such as memory device 105, and provides alerts, as described above, without requiring a secondary device 103 or other additional processor to interpret raw biological data. For example, a wearer of the cap 112 may wear the cap 112 and the biosensor assembly 101 may provide alerts regarding the physiological condition of the body of the wearer in remote locations, where the wearer is not in communication with any wireless networks or it is impractical for him/her to carry any other device, for instance during exercise. The biosensor assembly 101 may be configured, via its controlling unit 108, for example, to upload the stored data to the secondary device 103, after the monitored activity has terminated for reporting purposes only.

In some embodiments, the secondary device 103 may comprise a network server in communication with a wireless network that is accessible by the biosensor assembly 101 through its wireless communication functionality. The biosensor assembly 101 may upload the data, event history, and other information stored in the memory 105 to the network server for later retrieval and analysis.

The systems 100 and 100′, shown and described above, may also be suitable for monitoring children. In one scenario of use of the system 100 shown in FIGS. 1 and 2, a caregiver carries the secondary device 100 and receives alerts from the biosensor assembly 101 attached to a child's head covering, such as the cap 112, while in the range of wireless communication capability. In one scenario of use of the system 100′ shown in FIG. 4, a caregiver is close enough to a child wearing a head covering having the biosensor assembly 101 coupled to it to hear audible alerts generated by the biosensor assembly 101.

Having thus described the present invention by reference to certain of its preferred embodiments, it is noted that the embodiments disclosed are illustrative rather than limiting in nature and that a wide range of variations, modifications, changes, and substitutions are contemplated in the foregoing disclosure and, in some instances, some features of the present invention may be employed without a corresponding use of the other features. Many such variations and modifications may be considered desirable by those skilled in the art based upon a review of the foregoing description of preferred embodiments. Accordingly, it is appropriate that the appended claims be construed broadly and in a manner consistent with the scope of the invention.

1. A biosensor assembly configured to monitor biological data of a living body, the assembly comprising:
a first sensor configured to be coupled to a wearable item, wherein the first sensor is positionable on the wearable item to gather raw biological data from the body when the wearable item is worn by the body;
a controlling unit configured to be coupled to the wearable item and operationally connected to the first sensor, wherein the controlling unit is configured to receive the raw biological data gathered by the first sensor and to compute a representative value from the raw biological data;
a memory configured to be coupled to the wearable item and operationally connected to the controlling unit, wherein the memory is configured to store a first threshold value for access by the controlling unit; and
wherein the controlling unit is configured to compare the representative value with the first threshold value stored in the memory for characterizing a physiological condition of the body.

2. The assembly of claim 1, wherein the controlling unit is configured to generate a response signal based on the comparison of the first representative value with the first threshold value.

3. The assembly of claim 2, the assembly further comprising:
a signaling device operationally connected to the controlling unit, the signaling device configured to generate an alert to inform the user of a physiological condition of the living body; and
wherein the signaling device is configured to generate the alert based on the response signal received from the controlling unit.

4. The assembly of claim 2, wherein the signaling device is configured to be coupled to the wearable item.

5. The assembly of claim 2, wherein the signaling device is not coupled to the wearable item, and wherein the operational connection of the secondary device to the controlling unit is a wireless connection.

6. The assembly of claim 4, further comprising a presence sensor operationally connected to the controlling unit, wherein the controlling unit is configured to receive data from the presence sensor indicating that the wearable item is being worn by the body.

7. The assembly of claim 6, wherein the controlling unit is configured to receive data from the presence sensor to detect that the first sensor is positioned relative to the body to gather reliable raw biological data from the body.

8. The assembly of claim 4, wherein the first sensor is a temperature sensor configured to gather temperature data from the body, and wherein the first sensor gathers a first temperature reading.

9. The assembly of claim 8, wherein the controlling unit computes a first body temperature based on the first temperature reading, and wherein the controlling unit compares the first body temperature to the first threshold value to determine whether the first body temperature has exceeded the first threshold value.

10. The assembly of claim 9, wherein the controlling unit is configured to generate a response signal based on a comparison of the first body temperature to the first threshold value; wherein the controlling unit sends the response signal to a signaling device, and wherein the signaling device is configured to generate an audible alert to indicate the results of the comparison of the first body temperature to the first threshold value.

11. The assembly of claim 10, wherein the wearable item is a cap configured to be worn on the head of the body, and wherein the first sensor is coupled to the cap on an inner sweat band portion of the cap, and wherein, when the cap is worn by the body, the first sensor is positioned relative to the skin of the body to gather raw biological data from the body.

12. The assembly of claim 5, wherein the wireless connection between the secondary device and the controlling unit is Bluetooth.

13. A method configured to monitor biological data of a living body of a user of the method, the method comprising:
providing a first sensor affixed to a wearable item and positionable on the body for gathering raw biological data from the body;
providing a controlling unit having a memory, wherein the controlling unit is configured to interpret raw biological data received from the first sensor, wherein the controlling unit is operationally connected to the first sensor and affixed to the wearable item and wherein the memory is configured to store at least a first threshold value;
receiving, by the controlling unit, a first instance of raw biological data gathered by the first sensor;
computing, by the controlling unit, a first representative value from the first instance of raw biological data; and
comparing, by the controlling unit, the first representative value with the first threshold value stored in the memory for characterizing a physiological condition of the body.

14. The method of claim 13, further comprising:
providing a signaling device operationally connected to the controlling unit;
generating, by the controlling unit, a first response signal based on the comparison of the first representative value with the first threshold value;
sending the first response signal to the signaling device; and
generating, by the signaling device, a first alert to inform the user of a first physiological condition of the living body.

15. The method of claim 14, further comprising:
wherein the signaling device is affixed to the wearable item.

16. The method of claim 14, further comprising:
transmitting, via an antenna operationally connected to the controlling unit, the first response signal to the signaling device, wherein the operational connection between the controlling unit and the signaling device is a wireless connection.

17. The method of claim 15, further comprising:
receiving, by the controlling unit, a second instance of raw biological data gathered by the first sensor;
computing, by the controlling unit, a second representative value from the second instance of raw biological data;
comparing, by the controlling unit, the second representative value with a second threshold value stored in the memory;
generating, by the controlling unit, a second response signal based on the comparison of the second representative value with the second threshold value;
sending the second response signal to the signaling device;
generating, by the signaling device, a second alert to inform the user of a second physiological condition of the living body; and
wherein the second alert is configured to be distinguishable by the user from the first alert for distinguishing the first physiological condition from the second physiological condition of the body.

18. The method of claim 17, further comprising:
wherein the first sensor is a temperature sensor;
wherein the first representative value is a first body temperature of the body;
wherein the second representative value is second body temperature of the body;
wherein the first threshold value is a first threshold temperature;
wherein the second threshold value is a second threshold temperature;
wherein the first alert informs the user that the body temperature of the body has exceeded the first threshold temperature;
wherein the second alert informs the user that the body temperature of the body has exceeded the second threshold temperature.

19. The method of claim 18, further comprising:
wherein the second alert is configured to communicate to the user that exceeding the second temperature threshold is a greater threat to the health of the body than exceeding the first temperature threshold.
